(19) United States
(12) Patent Application Publication
(10) Pub. No.: US 2012/0004272 A1
(43) Pub. Date: Jan. 5, 2012

(54) BIOCIDAL COMPOSITION OF 2,6-DIMETHYL-M-DIOXANE-4-OL ACETATE AND METHODS OF USE

(76) Inventors: Kathy J. Ji, Shanghai (CN); Donald J. Love, Midland, MI (US); Jon B. Raymond, Buffalo Grove, IL (US)

(21) Appl. No.: 13/203,877

(22) PCT Filed: Mar. 26, 2009
(86) PCT No.: PCT/CN2009/071025

§ 371 Date: Aug. 30, 2011

(51) Int. Cl. A61K 31/421; A61P 31/00; A01N 43/32; A01P 1/00; A01N 43/76; A61K 31/357

(52) U.S. Cl. 514/376; 514/452

(57) ABSTRACT

Provided are compositions comprising 2,6-dimethyl-m-dioxane-4-ol acetate and a biocidal compound selected from the group consisting of: a biocidal oxazolidine; 1-(3-chloroallyl)-3,5,7-triaza-1-azoniaadamantane; and tris(hydroxymethyl)-nitromethane. The compositions are useful for controlling microorganisms in aqueous or water containing systems.

### FIELD OF THE INVENTION

The invention relates to biocidal compositions and methods of their use for the control of microorganisms in aqueous and water containing systems. The compositions comprise 2,6-dimethyl-m-dioxane-4-ol together with a second biocide.

### BACKGROUND OF THE INVENTION

Aqueous-based materials often need protection from microbial degradation and/or spoilage during shelf life and use. Preservatives are used to control microbial degradation and/or spoilage in aqueous materials, however, sometimes they are incapable of providing effective control over a wide range of microorganisms, even at high use concentrations. In addition, preservatives are often a costly component of a product. While combinations of different biocides are sometimes used to provide overall control of microorganisms in a particular end use environment, there is a need for additional combinations of microbicides having enhanced activity against various strains of microorganisms. There is also a need for combinations that utilize lower levels of individual microbicides for environmental and economic benefits.

### BRIEF SUMMARY OF THE INVENTION

In one aspect, the invention provides biocidal (i.e., preservative) compositions. The compositions are useful for controlling microorganisms in aqueous or water containing systems. The compositions of the invention comprise 2,6-dimethyl-m-dioxane-4-ol acetate together with a biocidal compound selected from the group consisting of: a biocidal oxazolidine; 1-(3-chloroallyl -3,5,7-triaza-1-azoniaadamantane; and tris(hydroxymethyl)-nitromethane.

In a second aspect, the invention provides a method for controlling microorganisms in aqueous or water containing systems. The method comprises treating the system with a biocidal composition as described herein.

### DETAILED DESCRIPTION OF THE INVENTION

As noted above, the invention provides biocidal compositions and methods of using them in the control of microorganisms. The compositions comprise 2,6-dimethyl-m-dioxane-4-ol acetate (“dimethoxane”) together with a biocidal compound selected from the group consisting of: a biocidal oxazolidine; 1-(3-chloroallyl -3,5,7-triaza-1-azoniaadamantane; and tris(hydroxymethyl)nitromethane. It has surprisingly been discovered that combinations of dimethoxane with other biocidal compounds as described herein, at certain weight ratios, are synergistic when used for microorganism control in aqueous or water containing media. That is, the combined materials result in improved biocidal properties than would otherwise be expected based on their individual performance. The observed synergy permits reduced amounts of the materials to be used to achieve acceptable biocidal properties, thus potentially reducing environmental impact and materials cost.

For the purposes of this specification, the meaning of “microorganism” includes, but is not limited to, bacteria, fungi, algae, and viruses. The words “control” and “controlling” should be broadly construed to include within their meaning, and without being limited thereto, inhibiting the growth or propagation of microorganisms, killing microorganisms, disinfection, and/or preservation.

In a first embodiment, the composition of the invention comprises 2,6-dimethyl-m-dioxane-4-ol acetate and a biocidal oxazolidine compound. Suitable oxazolidine compounds for use in this embodiment include, but are not limited to, monocyclic oxazolidines such as 4,4-dimethyoxazolidine (available from The Dow Chemical Company), N-methyl-1,3-oxazolidine, N-ethylol -1,3-oxazolidine, 5-methyl-1,3-oxazolidine, 4-ethyl-4-hydroxymethyloxazolidine, 4-ethyloxazolidine, and 4-methyl-4-ethyloxazolidine. 4,4-Dimethyoxazolidine is a preferred monocyclic oxazolidine.

Suitable oxazolidine compounds also include bicyclic oxazolidines, including 1 aza-3,7-bicyclo[3.3.0]octane optionally substituted with C₁-C₆ alkyl, C₁-C₆ alkoxy, or hydroxy(C₁-C₆ alkyl), such as 7-ethylbicyclooxazolidine (5-ethyl-1-aza-3,7-dioxabicyclo[3.3.0]octane) (available from The Dow Chemical Company), 5-hydroxymethoxymethyl-1-aza-3,7-dioxabicyclo[3.3.0]octane (available from International Specialty Products), 5-hydroxymethyl-1-aza-3,7-dioxabicyclo[3.3.0]octane (available fromInternational Specialty Products), 5-hydroxypoly(methyleneoxymethyl-1-aza-dioxabicyclo(3.3.0) octane (available from International Specialty Products), and 1-aza-3,7-dioxa-5-methylol-(3.3.0)-bicyclooctane. 7-Ethylbicyclooxazolidine is a preferred bicyclic oxazolidine.

Suitable oxazolidine compounds further include bisoxazolidines such as N,N-methylenebis(5-methyl-oxazolidine) (available from Halliburton) and bis-(4,4′-tetramethyl-1,3-oxazolidin-3-yl)-methane.

Suitable oxazolidine compounds additionally include polyoxazolidines.

Preferably, the 2,6-dimethyl-m-dioxane-4-ol acetate to oxazolidine weight ratio in the first embodiment of the invention is between about 1000:1 and about 1:1000, more preferably between about 500:1 and about 1:500, even more preferably between about 100:1 and about 1:100, and further preferably between about 20:1 and about 1:20. In a particularly preferred embodiment, the 2,6-dimethyl-m-dioxane-4-ol acetate to oxazolidine weight ratio is between about 13:1 and about 1:13.

Biocidal oxazolidine compounds for use in the invention are commercially available and/or can be readily prepared by those skilled in the art using well known techniques. Dimethoxane is commercially available.

In a second embodiment, the composition of the invention comprises 2,6-dimethyl-m-dioxane-4-ol acetate and 1-(3-chloroallyl -3,5,7-triaza-1-azoniaadamantane (“CTAC”). The CTAC compound may be the cis isomer, the trans isomer, or a mixture of cis and trans isomers. Preferably, it is the cis isomer or a mixture of the cis and trans isomers.

Preferably, the 2,6-dimethyl-m-dioxane-4-ol acetate to CTAC weight ratio in the second embodiment of the invention is between about 1000:1 and about 1:1000, more preferably between about 500:1 and about 1:500, even more preferably between about 100:1 and about 1:100, and further preferably between about 20:1 and about 1:20. In a particularly preferred embodiment, the 2,6-dimethyl-m-dioxane-4-ol acetate to CTAC weight ratio is between about 5:1 and about 1:1, even more preferably between about 1.6:1 and about 1:1.

CTAC is commercially available and/or can be readily prepared by those skilled in the art using well known techniques.

In a third embodiment, the composition of the invention comprises 2,6-dimethyl-m-dioxane-4-ol acetate and tris(hydroxymethyl)nitromethane. Preferably, the 2,6-dimethyl-m-dioxane-4-ol acetate to tris(hydroxymethyl)nitromethane weight ratio in this third embodiment is between about 1000:1 and about 1:1000, more preferably between about 500:1 and about 1:500, even more preferably between about 100:1 and about 1:100, and further preferably between about 20:1 and about 1:20. In a particularly preferred embodiment, the 2,6-dimethyl-m-dioxane-4-ol acetate to tris(hydroxymethyl)nitromethane weight ratio is between about 5:1 and about 1:1, even more preferably between about 3:1 and about 1.6:1.

Tris(hydroxymethyl)nitromethane is commercially available and/or can be readily prepared by those skilled in the art using well known techniques.

The compositions of the invention are useful at controlling microorganism growth in a variety of aqueous and water containing systems. Examples of such systems include, but are not limited to, paints and coatings, aqueous emulsions, latexes, adhesives, inks, pigment dispersions, household and industrial cleaners, detergents, dish detergents, mineral slurries polymer emulsions, caulks and adhesives, tape joint compounds, disinfectants, sanitizers, metalworking fluids, construction products, personal care products, textile fluids such as spin finishes, industrial process water (e.g. oilfield water, pulp and paper water, cooling water), oilfield functional fluids such as drilling muds and fracturing fluids, and fuels. Preferred aqueous systems are detergents, personal care, household, and industrial products, and paints/coatings. Particularly preferred are paints and coatings, detergents, and textile fluids such as spin finishes.

A person of ordinary skill in the art can readily determine, without undue experimentation, the concentration of the composition that should be used in any particular application. By way of illustration, a suitable actives concentration (total for both dimethoxane and the second biocide) is typically between 0.001 and 1 weight percent, preferably between 0.01 and 0.1 weight percent, based on the total weight of the aqueous or water containing system including the biocides.

The components of the composition can be added to the aqueous or water containing system separately, or preblended prior to addition. A person of ordinary skill in the art can easily determine the appropriate method of addition. The composition can be used in the system with other additives such as, but not limited to, surfactants, ionic/nonionic polymers and scale and corrosion inhibitors, oxygen scavengers, and/or additional biocides.

The following examples are illustrative of the invention but are not intended to limit its scope.

### EXAMPLES

### General.

Biocides. The following biocides are tested in these examples.

2,6-Dimethyl-m-dioxan-4-ol acetate (dimethoxane or “DMX”) is used as BIOBAN™ DXN, 87% active, available from The Dow Chemical Company.

4,4-Dimethyloxazolidine (“DMO”) is used as BIOBAN™ CS-1135, 78% active, available from The Dow Chemical Company.

7-Ethyl-bicyclooxazolidine (“EBCO”) is used as DOWICIL™ 96, 96% active, available from The Dow Chemical Company.

1-(3-Chloroallyl -3,5,7-triaza-1-azoniaadamantane choloride (“CTAC”) is used as DOWICIL™ 75, 64% active, available from The Dow Chemical Company.

2-Hydroxymethyl-2-nitro-1,3-propanediol (“TN”) is used as TRIS NITRO™, 50% active, available from The Dow Chemical Company.

Synergy Calculations. The reported synergy indexes are measured and calculated using the formula described below. In this approach, a synergy index of 1 indicates additivity. If the index is less than 1, synergy has occurred, while a synergy index greater than 1 indicates antagonism.

Synergy index=C_{A}/Cₐ+C_{B}/C_{b}
Cₐ=minimal concentration of antimicrobial A, alone, producing a 4 log₁₀ microbial kill
C_{b}=minimal concentration of antimicrobial B, alone, producing a 4 log₁₀ microbial kill

C_{A} and C_{B}=the concentrations of antimicrobials A and B, in combination, producing the required microbial kill (a 4 log₁₀ microbial kill unless indicated otherwise in a particular Example).

### Example 1

### Evaluation of Dimethoxane/Oxazolidines in Paint

In this Example, the antimicrobial profiles of 2,6-dimethyl-m-dioxan-4-ol (DMX), 4,4-dimethyloxazolidine (DMO), 7-ethyl-bicyclooxazolidine (EBCO) and combinations of DMX/DMO, DMX/EBCO are evaluated in a commercial (interior eggshell) water-based latex paint formulation (pH 7.4). The paint formulation is determined to be free of microbial contamination prior to initiation of preservative efficacy evaluations.

Experimental Setup. Tests are conducted in a 96-deep well block format using a total sample volume of 600 μl for all evaluations. In these samples, no more than 10% of the total volume consists of the biocide and organism solution and all non-matrix additions are normalized for all samples. Each experimental 96-well block contains biocide-treated samples and control samples which lack biocide.

Microorganisms. Twenty-four hour tryptic soy broth cultures are combined in equal parts for formulation inoculation at a final concentration of 5×10⁶ CFU/ml. Organisms are added to each sample of the 96-well block and mixed until homogenous. Additionally, bacterial challenges of the paint samples occur on days 0, 2, 7, and 14 of the 28-day test period. Organisms utilized: Pseudomonas aeruginosa (ATCC#15442), Pseudomonas aeruginosa (ATCC#10145), Enterobacter aerogenes (ATCC#13048), Escherichia coli (ATCC#11229), Klebsiella pneumoniae (ATCC#8308), Staphylococcus aureus (ATCC#6538), Salmonella choleraesuis (ATCC#10708).

Enumeration of Viable Organisms. Sample aliquots are removed, at predetermined time points, for the enumeration of surviving microorganisms. Numerical values in the data tables listed below represent the log₁₀ viable microorganisms recovered from individual samples at specific time points and biocide concentrations post microorganism addition. Biocide concentrations resulting in a ≧4 log₁₀ kill of microorganisms, as compared to the biocide-free control, are deemed a significant reduction of viable organisms and are subsequently used for calculating synergy index values. Results are shown in Tables 1-4.

TABLE 1
DAY 15 viable microorganism enumeration (post 4^{th} microbial challenge) for DMX and
DMO in paint.

| | DMO (ppm) | | | | | | | | DMX | DMO alone | |
|---|---|---|---|---|---|---|---|---|---|---|---|
| DMX (ppm) | 1560 | 1040 | 693 | 463 | 308 | 205 | 137 | 91 | alone | score | ppm |
| 1740 | 0 | 0 | 0 | 0 | 0 | 0 | 0 | 0 | 0 | 0 | 1560 |
| 1160 | 0 | 0 | 0 | 0 | 0 | 0 | 0 | 0 | 5 | 0 | 1040 |
| 773 | 0 | 0 | 0 | 0 | 0 | 0 | 0 | 0 | 8 | 0 | 693 |
| 516 | 0 | 0 | 0 | 0 | 0 | 0 | 0 | 0 | 7 | 0 | 463 |
| 344 | 0 | 0 | 0 | 0 | 0 | 0 | 0 | 0 | 8 | 0 | 308 |
| 229 | 0 | 0 | 0 | 0 | 0 | 0 | 0 | 3 | 8 | 6 | 205 |
| 153 | 0 | 0 | 0 | 0 | 0 | 0 | 0 | 7 | 7 | 7 | 137 |
| 102 | 0 | 0 | 0 | 0 | 0 | 0 | 5 | 5 | 7 | 8 | 91 |
| 0 | 7 | 7 | 7 | 7 | 7 | 7 | 7 | 7 | 7 | 7 | 0 |

TABLE 2
Synergy calculations for DMX and DMO in paint.

| | | | | DMX in | DMO in | |
|---|---|---|---|---|---|---|
| | | DMX | DMO | combi- | combi- | |
| | DMX:DMO | alone | alone | nation | nation | Synergy |
| Time | ratio | (ppm) | (ppm) | (ppm) | (ppm) | Index |
| Day 15 | 13:1 | 1740 | 308 | 1160 | 91 | .967 |
| Day 15 | 3:1 | 1740 | 308 | 229 | 91 | .427 |
| Day 15 | 1:1 | 1740 | 308 | 153 | 137 | .533 |
| Day 15 | 1:2 | 1740 | 308 | 102 | 205 | .725 |

*Biocide concentrations represented as ppm active DMX or DMO

As can be seen, 1740 ppm active 2,6-dimethyl-m-dioxan-4-ol (DMX), when used alone, is required to achieve a ≧4 log₁₀ microbial kill following four bacterial challenges. 308 ppm of 4,4-dimethyloxazolidine (DMO) is required to achieve a ≧4 log₁₀ microbial kill under the same testing conditions. Use of various concentration ratios of DMO and DMX results in a greater log₁₀ reduction in viable microorganisms under the same testing conditions indicating a synergistic combination of biocide actives.

TABLE 3
DAY 20 viable microorganism enumeration (post 4^{th} microbial challenge) for DMX/EBCO
in paint.

| | EBCO (ppm) | | | | | | | | DMX | EBCO alone | |
|---|---|---|---|---|---|---|---|---|---|---|---|
| DMX (ppm) | 1920 | 1280 | 853 | 569 | 379 | 252 | 169 | 112 | alone | score | ppm |
| 1740 | 0 | 0 | 0 | 0 | 0 | 0 | 0 | 0 | 4 | 0 | 1920 |
| 1160 | 0 | 0 | 0 | 0 | 0 | 0 | 1 | 7 | 8 | 7 | 1280 |
| 773 | 0 | 0 | 0 | 0 | 0 | 0 | 1 | 8 | 8 | 8 | 853 |
| 516 | 0 | 0 | 0 | 0 | 0 | 0 | 1 | 7 | 8 | 7 | 569 |
| 344 | 0 | 0 | 0 | 0 | 0 | 2 | 3 | 7 | 8 | 7 | 379 |
| 229 | 0 | 0 | 0 | 0 | 0 | 5 | 7 | 8 | 8 | 8 | 252 |
| 153 | 0 | 0 | 0 | 0 | 4 | 5 | 7 | 8 | 8 | 8 | 169 |
| 102 | 0 | 0 | 0 | 0 | 0 | 5 | 6 | 8 | 8 | 8 | 112 |
| 0 | 8 | 8 | 8 | 8 | 8 | 8 | 8 | 8 | 8 | 8 | 0 |

TABLE 4
Synergy calculations for DMX and EBCO in paint.

| | | | | DMX in | EBCO in | |
|---|---|---|---|---|---|---|
| | | DMX | EBCO | combi- | combi- | |
| | DMX:EBCO | alone | alone | nation | nation | Synergy |
| Time | ratio | (ppm) | (ppm) | (ppm) | (ppm) | Index |
| Day 20 | 7:1 | 1740 | 1920 | 1160 | 169 | .756 |
| Day 20 | 2:1 | 1740 | 1920 | 344 | 169 | .287 |
| Day 20 | 1:1 | 1740 | 1920 | 344 | 379 | .395 |
| Day 20 | 1:4 | 1740 | 1920 | 102 | 379 | .256 |
| Day 20 | 1:13 | 1740 | 1920 | 102 | 1280 | .726 |

*Biocide concentrations represented as ppm active DMX or EBCO

As can be seen from the data, 1740 ppm active 2,6-dimethyl-m-dioxan-4-ol (DMX), when used alone, is required to achieve a ≧4 log₁₀ microbial kill following four bacterial challenges. 1920 ppm of 7-ethyl-bicyclooxazolidine (EBCO) is required to achieve a ≧4 log₁₀ microbial kill under the same testing conditions. Use of various concentration ratios of EBCO and DMX results in greater log₁₀ reduction in viable microorganisms under the same testing conditions indicating a synergistic combination of biocide actives.

### Example 2

### Evaluation of Dimethoxane/Oxazolidines in Spinning Finish Emulsion

In this Example, the antimicrobial profiles of 2,6-dimethyl-m-dioxan-4-ol (DMX), 4,4-dimethyloxazolidine (DMO), 1-(3-chloroallyl)-3,5,7-triaza-1-azoniaadamantane choloride (CTAC), 2-hydroxymethyl-2-nitro-1,3-propanediol (TN) and combinations of DMX/DMO, DMX/CTAC, DMX/TN are evaluated in a spinning finish emulsion. The spinning finish emulsion is determined to be free of microbial contamination prior to initiation of preservative efficacy evaluations. The spinning finish emulsion is prepared by adding 1 part spinning finish oil to 9 parts distilled water followed by 30 minutes of mixing.

Experimental Setup. Tests are conducted in a 96-deep well block format using a total sample volume of 300 to 600 μl for all evaluations. In these samples, no more than 10% of the total volume consists of the biocide and organism solution and all non-matrix additions are normalized for all samples. Each experimental 96-well block contains biocide-treated samples and control samples which lack biocide.

Microorganisms. Twenty-four hour tryptic soy broth cultures are combined in equal parts for formulation inoculation at a final concentration of 5×10⁷ CFU/ml. Organisms are added to each sample of the 96-well block and mixed until homogenous.

Additionally, bacterial challenges of the spinning finish emulsion samples occur on days 0, 2, 7, and 14 of the 28-day test period. Organisms utilized: Pseudomonas aeruginosa (ATCC#15442), Pseudomonas aeruginosa (ATCC#10145), Enterobacter aerogenes (ATCC#13048), Escherichia coli (ATCC#11229), Klebsiella pneumoniae (ATCC#8308), Staphylococcus aureus (ATCC#6538), Salmonella choleraesuis (ATCC#10708).

Enumeration of Viable Organisms. Sample aliquots are removed, at predetermined time points, for the enumeration of surviving microorganisms. Biocide concentrations resulting in a ≧6 log₁₀ kill of microorganisms, as compared to the preservative (biocide)-free control, are deemed a significant reduction of viable organisms and are subsequently used for calculating synergy index values. Results are shown in Tables 5-7.

TABLE 5
DAY 27 synergy calculations (post 4^{th} microbial challenge) for DMX and
TN in spinning finish emulsion.

| | | | | DMX in | TN in | |
|---|---|---|---|---|---|---|
| | | DMX | | combi- | combi- | |
| | DMX:TN | alone | TN alone | nation | nation | Synergy |
| Time | ratio | (ppm) | (ppm) | (ppm) | (ppm) | Index |
| Day 27 | 1.6:1 | 1339 | 592 | 396 | 250 | .718 |
| Day 27 | 2:1 | 1339 | 592 | 515 | 250 | .807 |
| Day 27 | 3:1 | 1339 | 592 | 669 | 250 | .922 |

*ppm values represent the active biocide concentration necessary to achieve a ≧6 log₁₀ microbial kill at the specific time point.

1339 ppm active 2,6-dimethyl-m-dioxan-4-ol (DMX), when used alone, is required to achieve a ≧6 log₁₀ microbial kill following four bacterial challenges. 592 ppm of 2-hydroxymethyl-2-nitro-1,3-propanediol (TN) is required to achieve a ≧6 log₁₀ microbial kill under the same testing conditions. Use of various concentration ratios of TN and DMX results in a greater log₁₀ reduction in viable microorganisms under the same testing conditions, indicating a synergistic combination of biocide actives.

TABLE 6
DAY 27 synergy calculations (post 4^{th} microbial challenge) for DMX and
CTAC in spinning finish emulsion.

| | | | | DMX in | CTAC in | |
|---|---|---|---|---|---|---|
| | | DMX | CTAC | combi- | combi- | |
| | DMX:CTAC | alone | alone | nation | nation | Synergy |
| Time | ratio | (ppm) | (ppm) | (ppm) | (ppm) | Index |
| Day 27 | 1:1 | 1339 | 582 | 305 | 320 | .778 |
| Day 27 | 1.2:1 | 1339 | 582 | 396 | 320 | .846 |
| Day 27 | 1.6:1 | 1339 | 582 | 515 | 320 | .937 |

*ppm values represent the active biocide concentration necessary to achieve a ≧6 log₁₀ microbial kill at the specific time point.

1339 ppm active 2,6-dimethyl-m-dioxan-4-ol (DMX), when used alone, was required to achieve a ≧6 log₁₀ microbial kill following four bacterial challenges. 582 ppm of 1-(3-chloroallyl)-3,5,7-triaza-1-azoniaadamantane choloride (CTAC) was required to achieve a ≧6 log₁₀ microbial kill under the same testing conditions. Use of various concentration ratios of CTAC and DMX resulted in an equivalent or greater log₁₀ reduction in viable microorganisms under the same testing conditions indicating a synergistic combination of biocide actives.

TABLE 7
DAY 27 synergy calculations (post 4^{th} microbial challenge) for DMX and
DMO in spinning finish emulsion.

| | | | | DMX in | DMO in | |
|---|---|---|---|---|---|---|
| | | DMX | DMO | combi- | combi- | |
| | DMX:DMO | alone | alone | nation | nation | Synergy |
| Time | ratio | (ppm) | (ppm) | (ppm) | (ppm) | Index |
| Day 27 | 2:1 | 1339 | 355 | 396 | 195 | .845 |
| Day 27 | 2.6:1 | 1339 | 355 | 515 | 195 | .934 |

*ppm values represent the active biocide concentration necessary to achieve a ≧6 log₁₀ microbial kill at the specific time point.

1339 ppm active 2,6-dimethyl-m-dioxan-4-ol (DMX), when used alone, is required to achieve a ≧6 log₁₀ microbial kill following four bacterial challenges. 355 ppm of 4,4-dimethyloxazolidine (DMO) is required to achieve a ≧6 log₁₀ microbial kill under the same testing conditions. Use of various concentration ratios of DMO and DMX results in a greater log₁₀ reduction in viable microorganisms under the same testing conditions indicating a synergistic combination of biocide actives.

While the invention has been described above according to its preferred embodiments, it can be modified within the spirit and scope of this disclosure. This application is therefore intended to cover any variations, uses, or adaptations of the invention using the general principles disclosed herein. Further, the application is intended to cover such departures from the present disclosure as come within the known or customary practice in the art to which this invention pertains and which fall within the limits of the following claims.

1. A composition comprising:
2,6-dimethyl-m-dioxane-4-ol acetate; and
a biocidal compound selected from the group consisting of: a biocidal oxazolidine; 1-(3-chloroallyl)-3,5,7-triaza-1-azoniaadamantane; and tris(hydroxymethyl)nitromethane.

2. A composition according to claim 1 wherein the biocidal oxazolidine is a monocyclic oxazolidine.

3. A composition according to claim 2 wherein the biocidal oxazolidine is 4,4-dimethyoxazolidine, N-methyl-1,3-oxazolidine, N-ethylol -1,3- oxazolidine, 5-methyl-1,3-oxazolidine, 4-ethyl-4-hydroxymethyloxazolidine, 4-ethyloxazolidine, and 4-methyl-4-ethyloxazolidine, or mixtures of two or more thereof.

4. A composition according to claim 1 wherein the biocidal oxazolidine is a bicyclic oxazolidine.

5. A composition according to claim 4 wherein the biocidal oxazolidine is 1 aza-3,7-bicyclo[3.3.0]octane optionally substituted with C₁-C₆ alkyl, C₁-C₆ alkoxy, or hydroxy(C₁-C₆ alkyl.

6. A composition according to claim 5 wherein the biocidal oxazolidine is 7-ethylbicyclooxazolidine, 5-hydroxymethoxymethyl-1-aza-3,7-dioxabicyclo[3.3.0]octane, 5-hydroxymethyl-1-aza-3,7-dioxabicyclo3.3.0octane, 5-hydroxypoly(methyleneoxymethyl-1-aza-dioxabicyclo(3.3.0) octane, 1-aza-3,7-dioxa-5-methylol-(3.3.0)-bicyclooctane, or mixtures of two or more thereof.

7. A composition according to claim 1 wherein the biocidal oxazolidine is a bisoxazolidines.

8. A composition according to claim 7 wherein the biocidal oxazolidine is N,N-methylenebis(5-methyl-oxazolidine), bis-(4,4′-tetramethyl-1,3-oxazolidin-3-yl)-methane, or a mixture thereof.

9. A composition according to claim 1 wherein the biocidal oxazolidine is a polyoxazolidine.

10. A composition according to claim 1 wherein the biocidal compound is 1-(3-chloroallyl)-3,5,7-triaza-1-azoniaadamantane.

11. A composition according to claim 1 wherein the biocidal compound is tris(hydroxymethyl)-nitromethane.

12. A composition according to claim 1 wherein the 2,6-dimethyl-m-dioxane-4-ol acetate to biocidal compound weight ratio is between about 1000:1 and about 1:1000.

13. A method for controlling microorganisms in an aqueous or water containing system, the method comprising treating the system with a composition according to claim 1.

14. A method according to claim 13 wherein the aqueous or water containing system is selected from paints and coatings, aqueous emulsions, latexes, adhesives, inks, pigment dispersions, household and industrial cleaners, detergents, dish detergents, mineral slurries polymer emulsions, caulks and adhesives, tape joint compounds, disinfectants, sanitizers, spin finishes; metalworking fluids, construction products, personal care products, textile fluids such as spin finishes, industrial process water (e.g. oilfield water, pulp and paper water, cooling water), oilfield functional fluids such as drilling muds and fracturing fluids, and fuels.

15. A method according to 14 wherein the aqueous or water containing system is selected from personal care, household and industrial products, paints and coatings, and textile fluids.
